(19) United States
(12) Patent Application Publication
(10) Pub. No.: US 2006/0149275 A1
(43) Pub. Date: Jul. 6, 2006

(54) Apparatus and methods for tibial plateau leveling osteotomy

(76) Inventor: Calvin M. Cadmus, Oakdale, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/012,872

(22) Filed: Dec. 14, 2004

(51) Int. Cl. A61B 17/58

(52) U.S. Cl. 606/88

(57) ABSTRACT

A bone fixation plate and methods are disclosed for performing an optimally centered tibial plateau leveling osteotomy. The optimally centered osteotomy produces a curvilinear cut separating a section of the tibia comprising the tibial metaphysis from a proximal section of the tibia, such that the curvilinear cut has a radial center point above the tibial plateau along the long axis of the tibia, and preferably in relation to the intersection of the cruciate ligaments. The bone fixation plate has a distal expanse configured to overlay the proximal section of the tibia along the length of the tibia. The distal expanse has a plurality of mounting holes to fix the distal expanse to the proximal section of the tibia. The bone fixation plate also has a proximal expanse configured to overlay the tibial metaphysis, the proximal expanse having a plurality of mounting holes; radially spaced apart substantially equidistant from a center point.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to a bone fixation plate, and more particularly to a fixation plate for fixing the proximal tibia and metaphyseal tibia portions in a canine tibial osteotomy.

2. Description of Related Art

A common injury to the femoro tibial joint in the leg of the dog is a disruption or injury to the anterior, or cranial cruciate ligament. This damage leaves the dog's joint unstable and lameness and joint inflammation, i.e. arthritis, are common consequences. When the cruciate ligament is injured the natural slope of the tibial plateau, along with the forces exerted by the calf and quadriceps muscles cause the femur bone to slide down the top of the tibial plateau to thrust forward with each weight-bearing stride. This phenomenon, often referred to as tibial thrust, results in excessive wear of the cartilage of the joint.

One method of surgical treatment is to perform a cuneiform osteotomy.

This osteotomy allows rotation of a cylindrical portion of the metaphyseal region of the proximal tibia, also known as tibial plateau leveling osteotomy (TPLO). As shown in the prior art illustration in FIGS. 1 and 2, TPLO involves making a curvilinear cut 30 in the top of the tibia bone 32 (osteotomy) to include the tibial plateau 34 and tibial metaphysis 36. In the natural configuration, the plane 42 of the tibial plateau 34 is at an angle a (tibial plateau angle) with respect to horizontal. Once the curvilinear cut 30 is made, the tibial metaphysis section 36 is then rotated along the curved osteotomy while the tibia 32 is relatively stationary. This levels the slope of the plateau 42 such that the angle a of the plateau interfacing with the femur 38 is minimized or zero, as shown in FIG. 2.

After the fragments of bone are rotated to a predetermined correction angle, they are reattached by means of internal fixation devices, i.e. pins, wires and bone plates with screws are all appropriate. The bone plate and screws have many advantages and are usually considered the superior method. The effect of this correctional osteotomy is to alter or level the tibial surface of the femoro tibial joint. This essentially eliminates the utility of the cranial cruciate ligament such that the joint functions stably in absence of the ligament.

The bone plates that are currently available to accomplish TPLO fixation, for example those described in U.S. Pat. No. 5,304,180, have a triangular proximal portion having one circular and two elongate mounting holes to affix the plate to the tibial metaphysis 36. The bone plate also has a distal or shank portion to affix to the proximal portion of the tibia 32, and lock the orientation of the two segments. In this configuration, the round holes allow for the insertion of bone screws to affix the plate to the bone in a neutral or non-dynamic fashion. The elongated holes are intended to allow for fixation in a dynamic fashion. The elongate holes are intended to allow for compression or distraction of the two bone segments by positioning the drill hole in the bone at opposite ends of the elongate hole.

Current art bone plates, however, have lead to several problems. First, the large footprint and geometric configuration of the triangular proximal portion of the bone plate creates sub-optimal mounting configurations for the procedure. The fragment of bone (containing the tibial metaphysis 36) created by the curvilinear cut 30 is sometimes only slightly larger than the triangular portion of the corresponding fixation plate. This often leads to contouring or bending of the plate in a fashion to match the bone anatomy.

This contouring combined with the close proximity to the interior of the femoro tibial joint 40 can cause the misplacement of the proximal screw or screws into the joint. Referring to FIG. 1, the upper or most proximal portion 46 of the cranial tibial metaphysis 36 is composed of primarily cancellous or soft bone. In contrast, the more distal section 48 of the tibial metaphysis comprises a much higher concentration of denser cortical bone. Generally the concentration of cortical bone decreases and the cancellous bone increases as you move toward the tibial plateau and joint 40.

Because the cancellous bone is has softer, more porous properties, it tends to have less holding strength than its cortical counterpart. Thus, screws placed in the softer cancellous bone have a higher probability of loosening their purchase or stripping out. In addition, cancellous bone screws generally have more prominent screw threads for the same diameter thread, leading to a smaller inner screw diameter that weakens the screw in torsion and shear forces. Because of this screw design, cancellous bone screws are more susceptible to failure during installation.

Furthermore, the large footprint of the triangular proximal portion of current-art bone plates often drive the placement of the size and location of the osteotomy. The position of the osteotomy has been shown to affect postoperative Tibial Plateau Angle (TPA). It is important to note that the difference between the pre- and postoperative TPA must be sufficient to counter the cranial tibial force created by the damaged ligament.

Studies by Kowaleski, M. P. (Proceedings of Veterinary Orthopedic Society, 2004, Big Sky, Montana) have shown that if the osteotomy is centered horizontally on the long axis of the bone and vertically above the tibial metaphysis, ideally at the point where the two cruciate ligaments cross, no significant adverse biomechanical changes occur. When the osteotomy is not centered as such, there will be a shift in the long axis of the tibia, which may lead to undesirable post-operative anatomical changes such as genu veras and genu valgus may develop.

Because of the large triangular footprint of the current-art bone plates, the positioning of the osteotomy is driven largely by the need of the physician to create enough bone mass above the cut to allow for mounting of the proximal triangular plate. As a result, the osteotomy location center point 44 is off center well below the joint 40, as seen in FIG. 1 and U.S. Pat. Nos. 5,304,180 and 4,677,973. Thus, undesirable postoperative anatomical anomalies are more likely to develop.

Additionally, the geometric configuration of the mounting holes in the current-art bone plates generally only allow for one compression loading point. To promote healing, it is generally desirable to load the tibial metaphysis and proximal tibia in compression along the curvilinear osteotomy cut. With the current art mounting hole configurations, the location of the mounting holes is such that loading of second screw tends to relieve any loading of the first installed screw. Thus, generally the compressive force is only generated at one point.

In view of the foregoing deficiencies, is an object of the present invention to provide a TPLO bone plate configured to appropriately fit an optimally centered osteotomy.

It is a further object to match each osteotomy and clearly fit the parameters of the centered osteotomy.

It is yet another object of the present invention to provide a TPLO bone plate with a footprint configured to allow exclusive mounting of cortical bone screws.

It is yet a further object of the present invention to provide a TPLO bone plate that has a mounting configuration that allows for multiple loading points to distribute a compressive force on the osteotomy mating surfaces. At least some of these objectives will be met in the invention described hereinafter.

### BRIEF SUMMARY OF THE INVENTION

The present invention is directed to a new bone fixation plate to be used in an improved procedure for canine tibial plateau leveling osteotomy. The improved bone fixation plate promotes fastening of the cut bone sections resulting from an optimal centered osteotomy. With a centered osteotomy, the long axis of the tibia remains in the same (preoperative) position, and the surgeon may accurately achieve the desired postoperative tibial plateau angle to affect a counter to the cranial tibial thrust. Many anatomical changes (such as genu veras) are avoided with the centered osteotomy.

An aspect of the invention is a bone fixation plate for tibial plateau leveling osteotomy that produces a curvilinear cut separating a section of the tibia comprising the tibial metaphysis from a proximal section of the tibia. The bone fixation plate generally comprises a distal expanse configured to overlay the proximal section of the tibia along the length of the tibia. The distal expanse has a plurality of mounting holes for mounting to the proximal section of the tibia. The bone fixation plate also has a proximal expanse configured to overlay the tibial metaphysis. The proximal expanse has a plurality of mounting holes that are radially spaced apart substantially equidistant from a center point when the bone fixation plate is installed.

In one mode of the current aspect, the proximal expanse mounting holes are radially spaced apart substantially equidistant from the center point of the curvilinear cut when the bone fixation plate is installed.

In another mode of the current aspect, the proximal expanse mounting holes are configured to be substantially equidistant from the curvilinear cut center point when the curvilinear cut center point is located on the long axis of the tibia.

In another mode of the current aspect, the proximal expanse mounting holes are configured to be substantially equidistant from the curvilinear cut center point when the curvilinear cut center point is located above the tibial metaphysis.

In a preferred embodiment of the current mode, the curvilinear cut center point is located at a point along the long axis corresponding to an intersection of the anterior cruciate ligament and the posterior cruciate ligament emanating from the tibial metaphysis.

In another mode of the current aspect, the proximal expanse comprises a semicircular shape having a radial center point substantially coincident with the curvilinear cut center point. In one embodiment, the proximal expanse has a radius corresponding to the curvilinear cut radius. Preferably, the proximal expanse radius is substantially equal to the curvilinear cut radius.

In yet another mode of the current aspect, the mounting holes are substantially circular. Ideally, the proximal expanse comprises two or three mounting holes. In a preferred embodiment, all the proximal expanse mounting holes are configured to overlay the tibial metaphysis substantially adjacent to the curvilinear cut.

Alternatively, the proximal expanse mounting holes may be slotted. In such a configuration, each of the slotted mounting holes preferably have a wall substantially equidistant from the curvilinear cut.

Another aspect of the present invention is bone fixation plate for tibial plateau leveling osteotomy having a distal expanse configured to overlay the proximal section of the tibia along the length of the tibia. The distal expanse comprising a plurality of mounting holes for fastening the proximal section of the tibia. The bone fixation plate also has a proximal expanse configured to overlay the tibial metaphysis, wherein the proximal expanse has a plurality of mounting holes configured to overlay the tibial metaphysis substantially adjacent to the curvilinear cut.

In one mode of the current aspect, the proximal expanse mounting holes are arranged in a radial array. In a preferred embodiment, the radial array has a center point substantially coincident with the center point of the curvilinear cut. The curvilinear cut center point is preferably located above the tibial metaphysis.

In another mode of the current aspect, the proximal expanse is configured to overlay only over a cortical bone region substantially adjacent to the curvilinear cut. The proximal expanse may also have a curved shaped to match the radius of the curvilinear cut. In particular, the proximal expanse may have an outer radius substantially similar to the curvilinear cut radius. In addition, the proximal expanse may have an inner radius smaller than the outer radius sufficient to allow placement of the mounting holes. Ideally, the inner radius is configured such that the proximal expanse only overlays over the cortical bone region substantially adjacent to the curvilinear cut.

Yet another aspect of the invention is a bone fixation plate for tibial plateau leveling osteotomy, having a distal expanse configured to overlay the proximal section of the tibia along the length of the tibia, and a proximal expanse configured to overlay the tibial metaphysis, wherein the proximal expanse comprises a plurality circular mounting holes configured to independently generate a compressive force on the opposing surfaces of the tibial metaphysis section and the proximal tibia section created by the curvilinear cut.

In one mode of the current aspect, the proximal expanse circular holes are oversized and beveled such that a mounting screw may be positioned at one side of the oversized hole to generate the compressive force.

In another mode of the current aspect, the proximal expanse circular holes are arranged in a radial array. Preferably, the proximal expanse circular holes overlay a cortical bone region of the tibial metaphysis adjacent to the curvilinear cut. In one embodiment, the radial array has a center point located above the tibial metaphysis, and ideally is substantially coincidental with a center point of the curvilinear cut.

In yet another aspect of the current invention, a method is disclosed for performing a tibial plateau leveling osteotomy. The method comprises producing a curvilinear cut in the tibia adjacent to the tibial metaphysis, wherein the curvilinear cut separates a section of the tibia comprising the tibial metaphysis from a proximal section of the tibia, and wherein the curvilinear cut has a radius and a center point located above the tibial metaphysis. The tibial metaphysis section is then rotated about the center point to alter the angular orientation of the tibial metaphysis section with respect to the proximal tibia section. Finally, the tibial metaphysis section and the proximal tibia section are fixed to lock the altered angular orientation.

In one mode of the current aspect, fixing the tibial metaphysis section and the proximal tibia section comprises fastening a distal expanse of a bone fixation plate to the proximal tibia section with a first set of screws, and fastening a proximal expanse of a bone fixation plate to the tibial metaphysis section with a second set of screws. Preferably, the second set of screws consist of cortical bone screws.

In a preferred embodiment of the current mode, fastening a proximal expanse of a bone fixation plate comprises drilling a guide hole into the tibial metaphysis and installing a screw into the guide hole, wherein the guide hole is positioned such that installing the screw into the guide hole generates a compressive force on the tibial metaphysis and the proximal section of the tibia.

In a preferred variant of the current embodiment, the bone fixation plate comprises a plurality of oversized holes on the proximal expanse, and drilling a guide hole into the tibial metaphysis comprises positioning a drill bit off-center adjacent one side of one of the oversized holes, and drilling a guide hole adjacent the oversized hole such that a compressive force is generated when the screw is installed in the guide hole.

In yet another embodiment, the plurality of oversized holes are radially oriented on the proximal expanse of the bone fixation plate such that drilling a second guide hole adjacent a second circular hole generates an independent and additive compressive force when a second screw is installed into the second guide hole.

In yet another aspect of the invention, a drill guide for performing tibial plateau leveling osteotomy with a bone fixation plate is disclosed. The drill guide comprises a housing having a top surface and a bottom surface and a guide hole passing vertically through the housing from the top surface to the bottom surface. The guide hole is configured to accommodate a drill bit. In addition, the bottom surface of the housing comprises a cylindrical boss having a diameter configured to closely match the diameter of a mounting hole in the bone fixation plate, wherein the guide hole is oriented with respect to the cylindrical boss such that the drill bit is capable of creating a hole at a specific location with respect to the mounting hole.

In a preferred configuration, the guide hole is located adjacent to an outside wall of the cylindrical boss. In addition, the axis of the guide hole is preferably oriented substantially parallel to the axis of the cylindrical boss.

The drill guide may further comprise a handle connected to a side of the housing, wherein the handle is configured to allow the housing to be manually positioned and inserted into the mounting hole.

In a further aspect of the invention, a method is disclosed for performing a tibial plateau leveling osteotomy comprising producing a curvilinear cut separating a section of the tibia comprising the tibial metaphysis from a proximal section of the tibia, and rotating the tibial metaphysis section about the center point to alter the angular orientation of the tibial metaphysis section with respect to the proximal tibia section. The method further includes fastening a distal expanse of a bone fixation plate to the proximal tibia section with a first set of screws and fastening a proximal expanse of a bone fixation plate to the tibial metaphysis section by installing a second set of screws, wherein the second set of screws are eccentrically guided into a plurality of mounting holes that are positioned adjacent to and substantially equidistant from the curvilinear cut to generate a compressive force between the tibial metaphysis section and the proximal tibia section.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic view of a prior art TPLO procedure with a curvilinear cut under the tibial metaphysis.

FIG. 2 is a schematic view of a prior art TPLO procedure with the tibial plateau leveled.

FIGS. 3A and 3B illustrate an exemplary bone fixation plate of the present invention having a three-hole proximal expanse.

FIGS. 4A and 4B illustrate an exemplary bone fixation plate of the present invention having a two-hole proximal expanse.

FIGS. 5A and 5B illustrate an exemplary bone fixation plate of the present invention having a slotted three-hole proximal expanse

FIG. 6 is a perspective view of a proximal canine tibia after a centered TPLO has been performed.

FIG. 7 is a schematic view of a stifle joint having a curvilinear cut in accordance with the present invention.

FIG. 8 is a schematic view of a bone fixation plate of the present invention attached to lock the leveled orientation of the tibial metaphysis section and proximal tibia.

FIG. 9 is a cross-sectional view of the bone fixation plate of the present invention being fastened to the tibial metaphysis section and proximal tibia in accordance with the present invention.

FIG. 10A is a side view of a drill guide in accordance with the present invention.

FIG. 10B is a bottom view of the drill guide of FIG. 9A.

FIG. 10C is a top view of the drill guide of FIG. 9A.

### DETAILED DESCRIPTION OF THE INVENTION

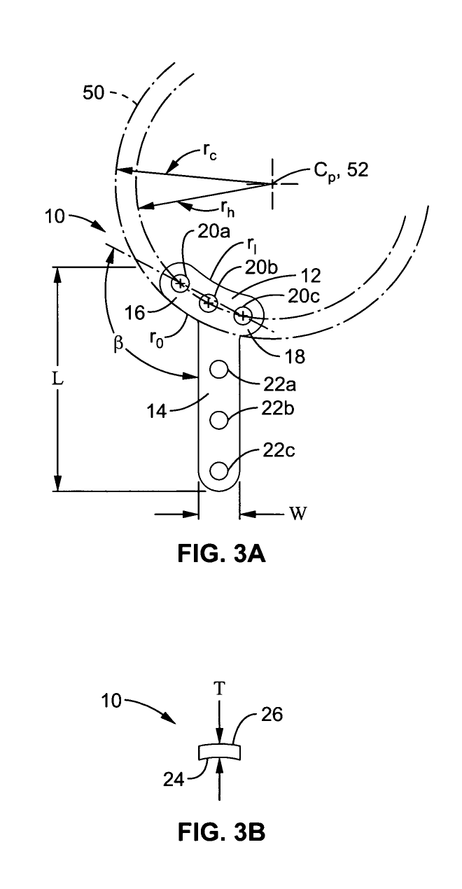

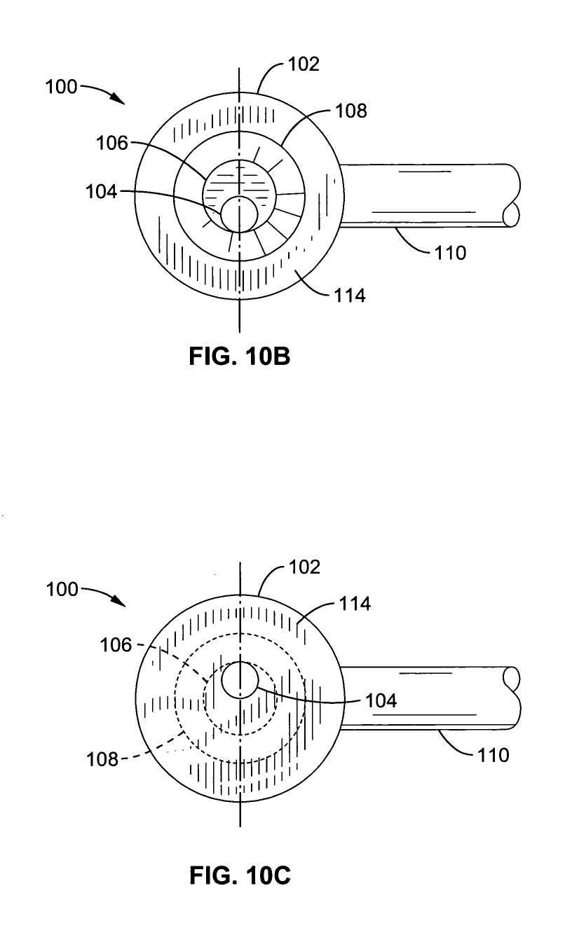

Referring more specifically to the drawings, for illustrative purposes the present invention is embodied in the apparatus generally shown in FIG. 3A through FIG. 10C. It will be appreciated that the apparatus may vary as to configuration and as to details of the parts, and that the method may vary as to the specific steps and sequence, without departing from the basic concepts as disclosed herein.

Referring to FIG. 3A, the bone plate 10 of the present invention may be subdivided into two portions: the proximal or top expanse 12 and the distal or bottom shank expanse 14. By way of example, the bone plate 10 is preferably made of a continuous piece of stainless steel, such as a sheet of 10-12 gauge SS 316 sheet metal. Other materials of similar stiffness and strength, such as titanium or titanium alloy may also be used.

The distal expanse 14 of bone plate 10 also has a plurality of mounting holes 22a,22b and 22c for mounting to the proximal tibia section 32. Generally, these holes are evenly spaced apart and are vertically oriented in a straight path to allow a stable mount on the relatively thin but elongate tibia 32. Distal expanse mounting holes 22a-c are also preferably centered between the width W of the distal expanse 14.

The proximal expanse 12 is configured to overlay the metaphysis 36 at the region closest to the curvilinear cut or osteotomy, which is characterized by dashed line 50. Proximal expanse 12 has a plurality of mounting holes 20a, 20b, and 20c to receive screws for fixation. In a preferred configuration, mounting holes 20a-c are positioned on the proximal expanse 12 in a radial pattern spaced substantially equidistant from center point Cₚ by a distance rₕ. This radial configuration allows the proximal expanse 12 to be fastened to the tibial metaphysis 36 at the closest possible mounting point to the osteotomy 50, where the cortical bone concentration is the highest to thus provide the optimal platform for steadfast mounting. As will be explained in further detail below, the radial alignment of mounting holes 20a-c also allows the proximal expanse to be mounted in such a way to evenly distribute a compressive load on the osteotomy 50 surfaces.

The anterior portion 16 of the proximal expanse is preferably semicircular and conforms to the curvilinear osteotomy 50. For example, the bottom side of anterior portion 16 and the smaller posterior portion 18 may be configured to coincide on a circle having an outer radius rₒ equal to or slightly less than the radius of the osteotomy path 50. The proximal expanse 12 preferably intersects the distal expanse 14 at an angle β. β is defined by the angle between a line bisecting distal expanse mounting holes 22a-c and a similar line bisecting the proximal expanse mounting holes 20a-c. The configuration of angle β is derived primarily from the geometry of the canine's anatomy such that the center point Cₚ of the proximal expanse mounting hole array aligns coincident, or nearly coincident, with the optimal center point 52 of centered osteotomy 50.

The top side of proximal expanse is generally closed out by a curved surface having an inner radius rₗ. Radius rₗ is generally smaller than the mounting hole array radius r_{H} and is preferably sized so that radius r_{H} is substantially equidistant between rₗ and rₒ. This distance between rₗ and rₒ will vary depending on the size of mounting holes 20a-20c, such that enough material is maintained on either side of the mounting holes to withstand the compressive forces generated by the mounting screws, as well as forces imparted from the canine's activity. This configuration creates the smallest possible vertical profile so that the bone plate 10 can be mounted higher up on the tibia in conformance with ideal osteotomy path 50 locations, as will be described in further detail below.

In alternative embodiments (not shown), the top and bottom surfaces (rₗ and rₒ) may comprise one or more linear edges to conform to the angle β and osteotomy path 50.

Referring to FIG. 3B, a bottom view of bone plate 10 is illustrated. The upper surface 26 and lower surface 24 are generally curved such that the majority of the surface of the plate is not in contact with the bone, thus preserving the bone's blood supply. The thickness T of the plate may vary depending on anatomy. For example, a 12 gauge sheet generally has a thickness of 0.105 inches, and a 10 gauge sheet generally has a thickness of 0.134 inches. Heavier or lighter gauge material may also be used depending on material selection and differing anatomies.

Because canine anatomy varies considerably for different breeds and sizes of dogs, the bone plate 10 may come in a number of different sizes and configurations. For example, the bone plate may be sized to accommodate a number of different sized osteotomy cuts, e.g. 18 mm, 24 mm, and 30 mm radius cuts. By way of example and without limitation, a plate with an 18 mm rₒ may have a β of 108°, L of approximately 1.57 in., W of approximately 0.315 in., 3.22 mm (0.1250 in.) mounting-hole diameters, and comprise 12 gauge SS sheet. Correspondingly, an exemplary plate with a 24 mm rₒ may have a β of 116°, L of approximately 2.20 in., W of approximately 0.4 in., 4.5 mm (0.1719 in.) mounting hole diameters, and comprise 10 gauge SS. An exemplary plate with a 30 mm rₒ may have a β of 113°, L of approximately 2.22 in., W of approximately 0.4 in., 4.5 mm (0.1719 in.) mounting hole diameters, and comprise 10 gauge SS. It will be appreciated that the above dimensions are representative of typical canine anatomies, and can be varied to accommodate larger or smaller anatomies.

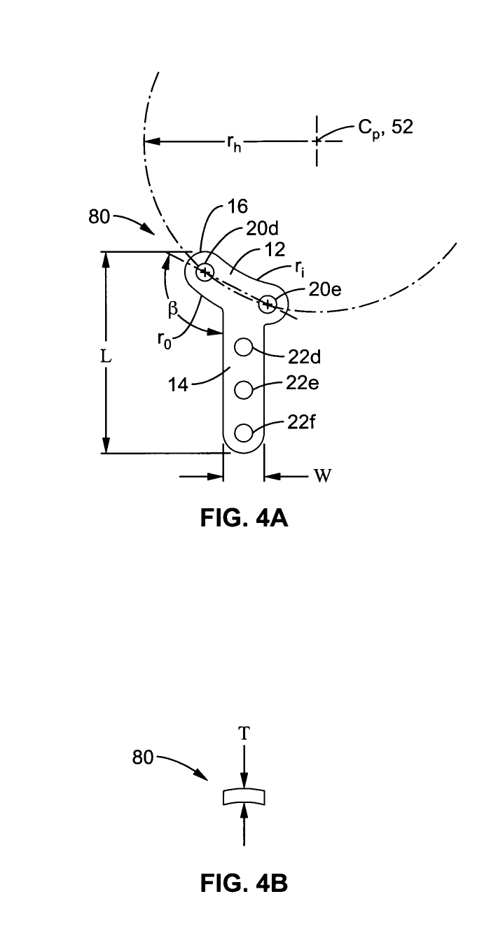

Thus, a surgeon may have a kit of various sized bone plates (i.e. 18 mm, 24 mm and 30 mm) at hand to accommodate varying anatomies. In addition, a kit may also comprise mirror image versions (not shown) of the bone plates of FIGS. 3A-4B so that left and right leg procedures may be performed.

Now referring to FIGS. 4A and 4B, an alternative bone plate 80 of the present invention may comprise a two-hole mounting configuration for the proximal expanse 12. Proximal expanse 12 has two mounting holes 20d and 20e to receive screws for fixation. Mounting holes 20d and 20e are positioned in a radial pattern spaced substantially equidistant from center point Cₚ by a distance rₕ.

The anterior portion 16 of the proximal expanse is semicircular with radius rₒ being substantially similar to the curvilinear osteotomy 50. The proximal expanse 12 preferably intersects the distal expanse 14 at an angle β. βis defined by the angle between a line bisecting distal expanse mounting holes 22d-22f and a similar line bisecting the proximal expanse mounting holes 20d and 20e.

By way of example and without limitation, a bone fixation plate 80 with a two-hole proximal expanse with an 18 mm rₒ may have a β of 103°, L of approximately 1.375 in., W of approximately 0.315 in., 3.22 mm (0.1250 in.) mounting-hole diameters, and comprise 12 gauge SS sheet. Correspondingly, an exemplary plate with a 24 mm rₒ may have a β of 116°, L of approximately 1.875 in., W of approximately 0.4 in., 4.5 mm (0.1719 in.) mounting hole diameters, and comprise 10 gauge SS.

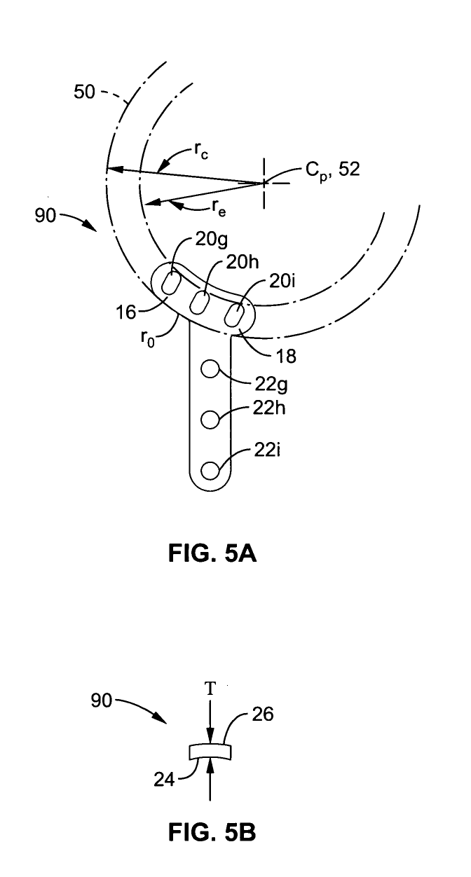

Now referring to FIGS. 5A and 5B, an alternative bone plate 90 of the present invention may comprise a slotted-hole mounting configuration for the proximal expanse 12. Proximal expanse 12 has three slotted mounting holes 20g-20i to receive screws for fixation. The mounting holes 20g-20i are positioned in a radial pattern such that the proximal walls of the slotted mounting holes are spaced substantially equidistant from center point Cₚ by a distance rₑ. The slotted holes preferably have the same length, but may be different sizes as long as the proximal walls of the holes lie equidistant from the center point Cₚ. Slotted holes 20g-20i may be oriented parallel to each other, with the center hole directed in line with center point Cₚ, as shown in FIG. 5A. Alternatively, slotted holes 20g-20i may be oriented such that they are all directed radially inward toward center point Cₚ. Slotted holes may also be used with the 2-hole proximal plate design shown in FIG. 4A.

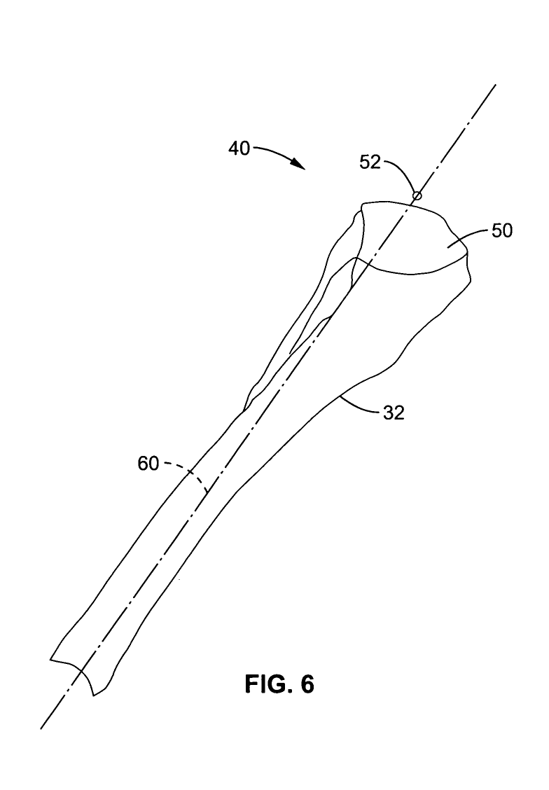

FIGS. 6-9 illustrate an exemplary method of performing a centered tibial plateau leveling osteotomy according to the present invention. FIG. 6 shows a perspective view of a canine tibia sans the tibial metaphysis after a centered osteotomy has been performed. The osteotomy 50 generally comprises a curvilinear or cylindrical cut substantially perpendicular to the sagittal plane.

The center-point location 52 of the osteotomy 50 on the tibia 32 is critical to the success of the procedure, as a non-centered osteotomy may lead to postoperative complications. The optimal location of the osteotomy center point 52 lies on the long axis 60 of the tibia 32, above the tibial plateau.

The long axis 60 generally passes through the midline of the tibia 32 along its length, and can be identified by anatomical markers 80 (intersection of the tibial spines 81) and 82 (center of ankle joint).

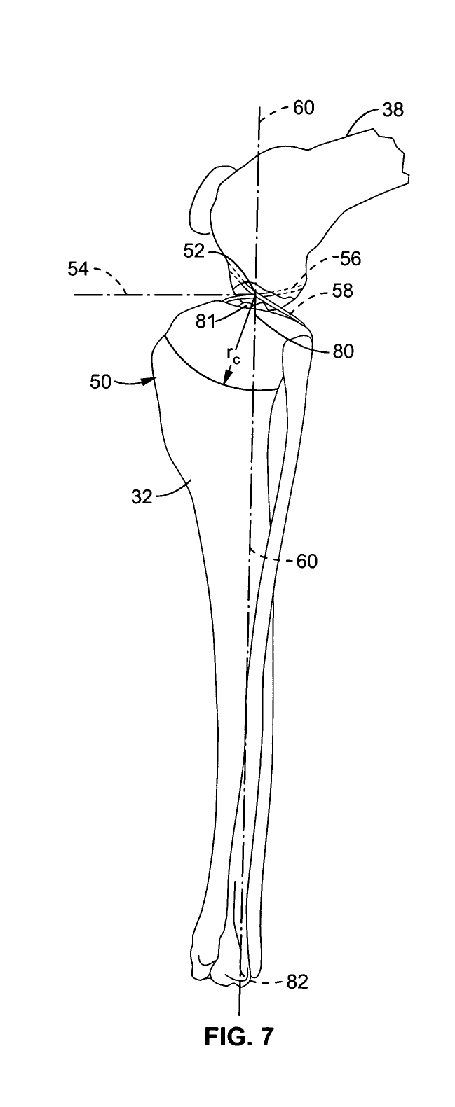

As illustrated in a partially cut-out schematic view of a canine stifle (knee) joint 40 shown in FIG. 7, the vertical position may be further defined by locating the intersection of the anterior (cranial) cruciate ligament 56 and the posterior (caudal) cruciate ligament 58. From this intersection, line 54 may be drawn out normal to the long axis 60 to define the center point location 52. Where one or more of the cruciate ligaments are torn, the vertical location may be approximated by assessing where the ligaments would have passed via anatomical landmarks such as the endpoints of the ligaments.). In many cases, the intersection of the cruciate ligaments will be closely related to the intersection of the tibial spines 81. It will be appreciated to one skilled in the art that the anatomical configuration illustrated in FIG. 7 is merely illustrative of a sample anatomy, and the anatomical configuration may vary greatly from patient to patient.

With the osteotomy center point 52 located, the veterinary surgical operation is performed to level the tibial plateau 34. Appropriate surgical exposure to the medial aspect of the proximal tibia 32 is accomplished. Disruption of musculature from the lateral aspect of the bone is not recommended. Slight elevation of the popliteus muscle on the caudal tibia is helpful. A curvilinear osteotomy 50 is made with its center point 52 along the long axis 60 of the tibia and at the level of the intersection of the cruciate ligaments.

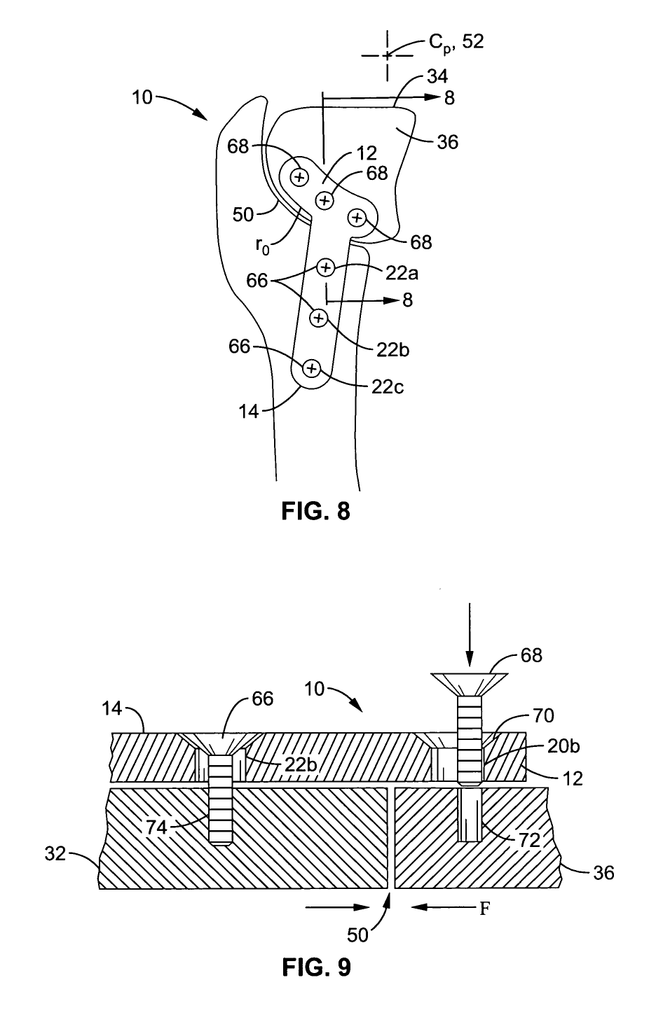

Referring now to FIG. 8, the newly created metaphyseal segment 36 is then rotated counter-clockwise relative to the proximal tibia 32 to a predetermined angle, thereby creating a smaller or more level angle to the tibial plateau 34. This in turn counters the cranial unstable movement of the tibia caused by the absence or damage to the cranial cruciate ligament. The rotated segment 36 is held in temporary alignment by a fixation pin (not shown).

With the tibial plateau 34 leveled, the two segments are then locked in place with bone fixation plate 10. The distal expanse 14 of the bone plate 10 is first affixed to the proximal tibial section 32. The bone plate 10 is positioned at the appropriate location on the tibia, preferably such that center point Cₚ substantially coincides with the osteotomy center point 52, with the external radial rₒ of the proximal expanse 12 closely following the contours of the curvilinear cut 50.

Once the bone plate 10 is properly positioned, guide holes 74 are created at the distal expanse hole (22a-c) locations, as shown in FIG. 8. The surgeon has the ability to select the position of the guide holes in the bone relative to the mounting holes in the bone plate 10. This allows for the metaphyseal segment 36 of bone to be driven into compression, distraction or neither compression nor distraction.

The distal expanse 14 is preferably mounted in an unloaded state, thus the guide holes 74 are located and drilled at the center points of the of the mounting holes 22a-c. Bone screws 66 are then installed into the bone of the proximal tibia 32 and then tightened to lock the in the distal expanse 14 in place. The top of the mounting holes 22a-c are countersunk or beveled with taper 70 to match the head of screws 66. The beveled holes assure that the distal expanse 14 does not translate with respect to the bone once it is fastened.

After fixation of the distal expanse 14 of the bone plate 10 to the proximal tibia 32, eccentric guide holes 72 are drilled into the remaining proximal expanse mounting hole (20a-c) locations. The eccentric placement of the guide holes creates a compressive force on the osteotomy 50 surfaces to speed osteosynthesis and bone healing.

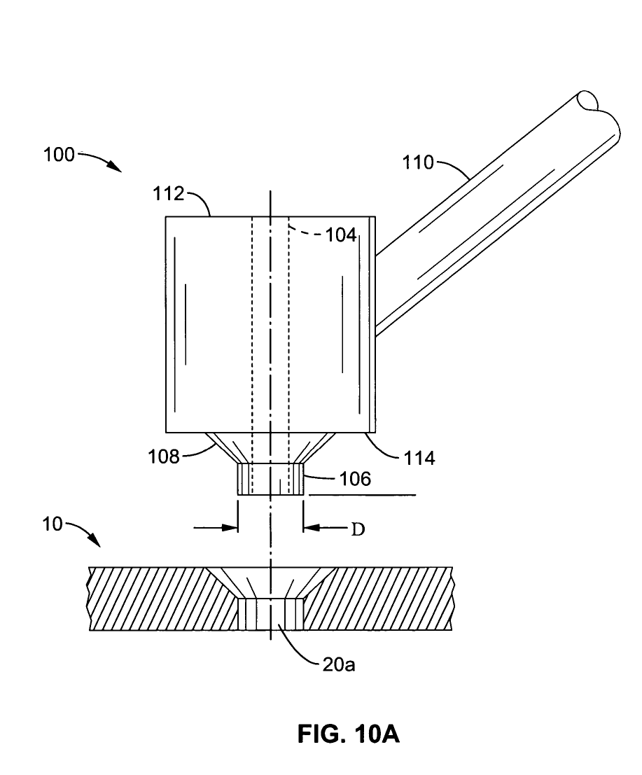

As illustrated in the cross-sectional view of FIG. 9, each of the guide holes 72 are drilled such that the resulting guide hole is as far as possible from the center of the mounting hole and abutting one wall of the mounting hole 20b. To affect a compressive force, the guide holes 72 are preferably placed on the mounting-hole wall furthest from the curvilinear cut 50.

It will be appreciated that with minimal modification, the present invention may be used for a variety of different procedures where it is necessary to apply a compressive force to promote healing of two adjacent bone segments, whether on canines, other animals, or humans. The bone fixation plate and methods illustrated in FIG. 3A-FIG. 9 are ideally configured for applying a compressive force to a TPLO curvilinear cut. However, the fixation plate may be sized to accommodate other procedures resulting in a curvilinear cut separating two bone segments.

Furthermore, the mounting patterns on either the proximal expanse or the distal expanse may be configured to accommodate linear cuts, wherein one of the mounting patterns (e.g. proximal expanse mounting holes) are aligned in an array parallel to the cut such that the center points of each mounting hole are each equidistant from the cut. For example, the line passing through the center points of mounting holes 20d and 20e of fixation plate 80 shown in FIG. 4A may be parallel to a linear cut path (not shown).

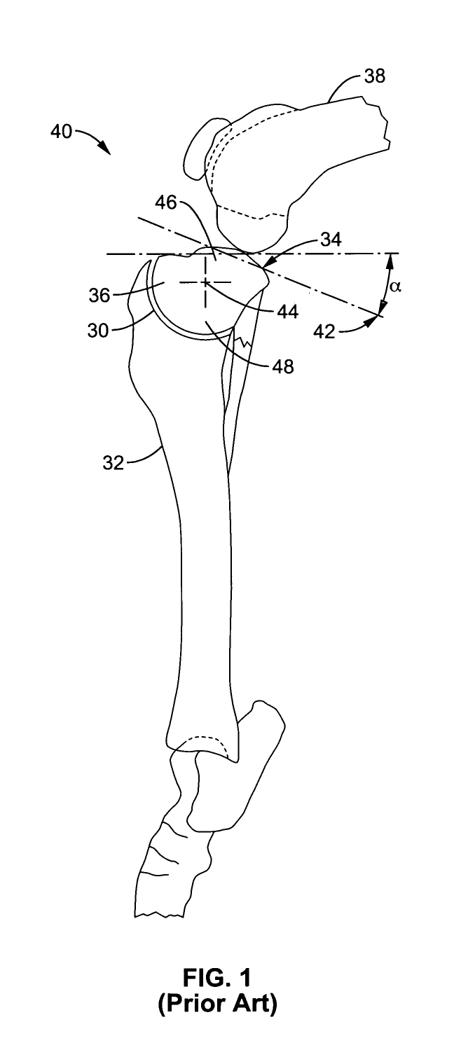

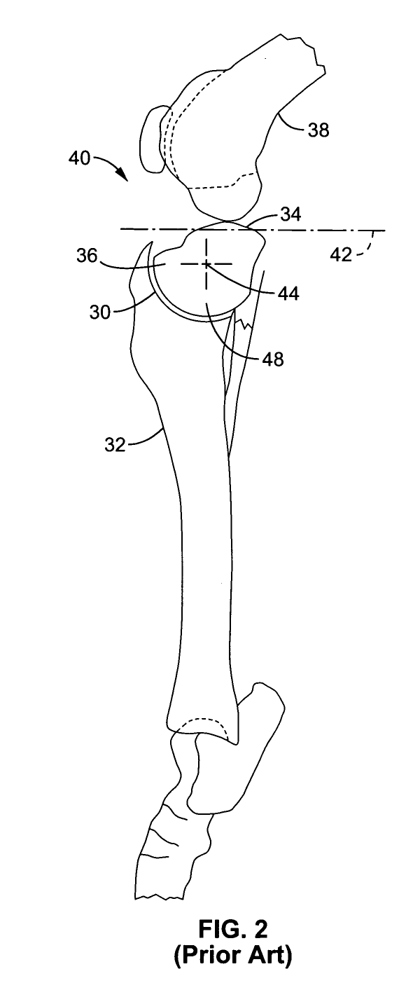

The eccentric placement of guide holes 72 may be facilitated by use of drill guide 100, as illustrated in FIGS. 1A-C. Drill guide 100 comprises a housing 102 fastened to a handle 110. A guide hole 104 vertically passes through the top 112 and bottom 114 surfaces of the housing. The guide hole has a diameter that closely matches the diameter of the intended drill bit for drilling bone guide holes 72. The bottom surface 114 of the housing 102 has a cylindrical boss 106 that has a diameter D closely matching the diameter of the mounting holes 20a-c. The boss 106 also has a tapered section 108 that closely matches the beveled surface of mounting holes 20a-c. The central axis of the boss 106 is preferably parallel to the axis of the guide hole 104 such that the drill will be guided perpendicular to the bone plate 10 and therefore in the same plane as the osteotomy 50 when the boss is placed in any of holes 20a-c.

Preferably, guide hole 104 is eccentrically located such that one side of the guide hole wall comes in point contact, or close to point contact, with one side of the outside wall of the boss 106. This eccentric positioning ensures that the bone guide hole 72 matches up with one wall of the mounting hole 20a-c.

A separate drill guide (not shown) may also be used of placement of the distal screws 66 into the distal expanse 14. In this configuration, the guide hole 104 is centered on boss 106 with their axes substantially coincident. This allows for centered placement of screws 66 in the distal expanse holes 22a-c for unloaded mounting.

Once the guide holes 72 are drilled, screws 68 are introduced into the guide holes 72 and brought to the level of the plate. The screws 68 are then tightened one quarter turn at a time, alternating between the three (or two screws for bone plate 80) screws. As screws 68 are tightened, the beveled surface 70 and matching screw head of screws 68 along with the eccentric positioning of the guide holes 72 shifts the metaphyseal segment 36 of the bone toward the proximal tibia 32, and thus generating a compressive force F into the proximal tibial segment 32 along the majority of the length of the cuneiform osteotomy.

Because the drilled guide holes 72 and mounting holes 20a-c and are also on a radius parallel to or coincident with the curvilinear osteotomy, the compressive forces generated by each individual screw 68 are additive, and help to evenly distribute the compressive load across the osteotomy 50 surfaces. This provides a dramatic improvement over existing art bone plates wherein the non-radial placement of the mounting holes creates a fulcrum effect that essentially unloads the first mounted screw upon loading of the second screw.

Because screws 66 and 68 are all placed in regions of relatively high density of cortical bone, cortical bone screws may be used. Cortical bone screws are preferable rather than cancellous bone screws, which tend to be weaker and have less holding strength.

After the proximal expanse screws 68 are tightened, the soft tissues and skin are then closed in a routine manner.

The advantages of the present invention over previously existing bone plates designed for this surgical procedure are numerous. The improved footprint of the mounting plate allows for the osteotomy to be created at the optimal location. All screws are placed in distal metaphysis and proximal tibia thusly decreasing the probability of damage to the interior of the stifle joint. Stronger cortical cone screws are placed in stronger cortical bone for a more secure mount. Compression is accomplished by multiple radially positioned metaphyseal screws and therefore compress along an increased segment of the osteotomy.

Although the description above contains many details, these should not be construed as limiting the scope of the invention but as merely providing illustrations of some of the presently preferred embodiments of this invention. For example, the bone fixation techniques and bone plate may be sized to accommodate a number of different procedures on various animals and/or humans, especially where the fixation plate is used to apply a compressive force to bone segments separated by a curvilinear cut. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A bone fixation plate for tibial plateau leveling osteotomy, said osteotomy producing a curvilinear cut separating a section of the tibia comprising the tibial metaphysis from a proximal section of the tibia, the curvilinear cut having a radius and a center point, the bone fixation plate comprising:
a distal expanse configured to overlay the proximal section of the tibia along the length of the tibia;
the distal expanse comprising a plurality of mounting holes; and
a proximal expanse configured to overlay the tibial metaphysis;
the proximal expanse comprising a plurality of mounting holes;
wherein the proximal expanse mounting holes are radially spaced apart substantially equidistant from a center point.

2. A bone fixation plate as recited in claim 1, wherein the proximal expanse mounting holes are radially spaced apart substantially equidistant from the center point of the curvilinear cut when the bone fixation plate is installed.

3. A bone fixation plate as recited in claim 1:
wherein the tibia comprises a long axis along its length;
wherein the curvilinear cut center point is located on the long axis; and
wherein proximal expanse mounting holes are configured to be substantially equidistant from the curvilinear cut center point when the bone fixation plate is installed.

4. A bone fixation plate as recited in claim 3:
wherein the curvilinear cut center point is located above the tibial metaphysis; and
wherein the proximal expanse mounting holes are configured to be substantially equidistant from the curvilinear cut center point.

5. A bone fixation plate as recited in claims 4, wherein the curvilinear cut center point is located at a point along the long axis corresponding to an intersection of the anterior cruciate ligament and the posterior cruciate ligament emanating from the tibial metaphysis.

6. A bone fixation plate as recited in claim 2, wherein the proximal expanse comprises a semicircular shape having a radial center point substantially coincident with the curvilinear cut center point.

7. A bone fixation plate as recited in claim 6, wherein the proximal expanse has a radius corresponding to the curvilinear cut radius.

8. A bone fixation plate as recited in claim 7, wherein the proximal expanse has a radius substantially equal to the curvilinear cut radius.

9. A bone fixation plate as recited in claim 1, wherein the mounting holes are substantially circular.

10. A bone fixation plate as recited in claim 1:
wherein the mounting holes are slotted; and
wherein the slotted mounting holes each have a wall substantially equidistant from the curvilinear cut.

11. A bone fixation plate as recited in claim 1, wherein the proximal expanse comprises two mounting holes.

12. A bone fixation plate as recited in claim 1, herein the proximal expanse comprises three mounting holes.

13. A bone fixation plate as recited in claim 1, wherein all the proximal expanse mounting holes are configured to overlay the tibial metaphysis substantially adjacent to the curvilinear cut.

14. A bone fixation plate for tibial plateau leveling osteotomy, said osteotomy producing a curvilinear cut separating a section of the tibia comprising the tibial metaphysis from a proximal section of the tibia, the bone fixation plate comprising:
a distal expanse configured to overlay the proximal section of the tibia along the length of the tibia;
the distal expanse comprising a plurality of mounting holes; and
a proximal expanse configured to overlay the tibial metaphysis;
the proximal expanse comprising a plurality of mounting holes;
wherein all of the proximal expanse mounting holes are configured to overlay the tibial metaphysis substantially adjacent to the curvilinear cut.

15. A bone fixation plate as recited in claim 14, wherein the proximal expanse mounting holes are arranged in a radial array.

16. A bone fixation plate as recited in claim 15:
wherein the curvilinear cut has a radius and a center point; and
wherein the radial array has a center point substantially coincident with the center point of the curvilinear cut.

17. A bone fixation plate as recited in claim 16, wherein the curvilinear cut center point is located above the tibial metaphysis.

18. A bone fixation plate as recited in claim 14, wherein the proximal expanse is configured to overlay only over a cortical bone region substantially adjacent to the curvilinear cut.

19. A bone fixation plate as recited in claim 14:
wherein the curvilinear cut has a radius and a center point; and
wherein the proximal expanse has a curved shape to match the radius of the curvilinear cut.

20. A bone fixation plate as recited in claim 19, wherein the proximal expanse has an outer radius that is substantially aligned with a portion of the curvilinear cut.

21. A bone fixation plate as recited in claim 20, wherein the proximal expanse has an inner radius proximal to the outer radius sufficient to allow placement of the proximal expanse mounting holes.

22. A bone fixation plate as recited in claim 21, wherein the inner radius is configured such that the proximal expanse only overlays over a cortical bone region substantially adjacent to the curvilinear cut.

23. A bone fixation plate for tibial plateau leveling osteotomy, said osteotomy producing a curvilinear cut separating a section of the tibia comprising the tibial metaphysis from a proximal section of the tibia, the bone fixation plate comprising:
a distal expanse configured to overlay the proximal section of the tibia along the length of the tibia;
the distal expanse comprising a plurality of circular mounting holes; and
a proximal expanse configured to overlay the tibial metaphysis;
the proximal expanse comprising a plurality of circular mounting holes;
wherein the proximal expanse circular mounting holes are configured to independently generate a compressive force on the opposing surfaces of the tibial metaphysis section and the proximal tibia section created by the curvilinear cut.

24. A bone fixation plate as in claim 23, wherein the proximal expanse circular holes are oversized and beveled such that a mounting screw may be positioned at one side of the oversized hole to generate the compressive force.

25. A bone fixation plate as in claim 23, wherein the proximal expanse circular holes are arranged in a radial array.

26. A bone fixation plate as in claim 23, wherein the proximal expanse circular holes overlay a cortical bone region of the tibial metaphysis.

27. A bone fixation plate as in claim 25, wherein the radial array has a center point located above the tibial metaphysis.

28. A bone fixation plate as in claim 27, wherein the radial array center point is substantially coincidental with a center point of the curvilinear cut.

29-44. (canceled)

45. A bone fixation plate as in claim 14, wherein at least a portion of each of the proximal expanse mounting holes lie on a straight line passing through the proximal expanse.

46. A bone fixation plate as in claim 14, wherein at least a portion of each of the proximal expanse mounting holes lie on a radial line passing through the proximal expanse, said radial line having a center point substantially coincidental with a center point of the curvilinear cut.

47. A bone fixation plate as in claim 21, wherein the proximal expanse mounting holes each have a wall substantially equidistant to the inner radius of the proximal expanse.

48. A bone fixation plate for tibial plateau leveling osteotomy, said osteotomy producing a curvilinear cut separating a section of the tibia comprising the tibial metaphysis from a proximal section of the tibia, the bone fixation plate comprising:
a distal expanse configured to overlay the proximal section of the tibia along the length of the tibia;
the distal expanse comprising a plurality of mounting holes; and
a proximal expanse configured to overlay the tibial metaphysis;
the proximal expanse comprising a plurality of mounting holes;
wherein at least a portion of each of the proximal expanse mounting holes lie on a radial line passing through the proximal expanse, said radial line having a center point substantially coincidental with a center point of the curvilinear cut.

49. A bone fixation plate as recited in claim 48, wherein at least a portion of each of the proximal expanse mounting holes lie on a straight line passing through the proximal expanse.

50. A bone fixation plate as recited in claim 49, wherein said straight line is oriented at an angle β with respect to a line passing through a center point of each of the distal expanse mounting holes, wherein β is an angle greater than 90°.

51. A bone fixation plate as recited in claim 49, wherein β is an angle ranging from 103° to 116°.

52. A bone fixation plate for tibial plateau leveling osteotomy, said osteotomy producing a curvilinear cut separating a section of the tibia comprising the tibial metaphysis from a proximal section of the tibia, the curvilinear cut having a radius and a center point, the bone fixation plate comprising:
a distal expanse configured to overlay the proximal section of the tibia along the length of the tibia;
the distal expanse comprising a plurality of mounting holes; and
a proximal expanse configured to overlay the tibial metaphysis substantially adjacent the curvilinear cut;
the proximal expanse comprising a plurality of mounting holes;
wherein the proximal expanse comprises a radial arm that substantially follows the contour of the curvilinear cut.

53. A bone fixation plate as recited in claim 52, wherein:
the proximal expanse is bounded by an top side and a bottom side;
wherein at least a portion of the bottom side is configured to lie adjacent the curvilinear cut; and
wherein the top side follows a path substantially similar to the curvilinear cut to minimize a vertical profile of the proximal expanse from the curvilinear cut, while still allowing placement of the proximal expanse mounting holes between the top side and bottom side.

54. A bone fixation plate as recited in claim 53, wherein the proximal expanse mounting holes are spaced substantially equidistant between the top side and the bottom side of the proximal expanse.

55. A bone fixation plate as recited in claim 53, wherein at least a portion of the bottom side is defined by an outer radius;
wherein said outer radius is substantially similar to the curvilinear cut radius.

56. A bone fixation plate as recited in claim 55, wherein at least a portion of the top side is defined by an inner radius;
wherein said inner radius is substantially similar to the curvilinear cut radius.
